(19) United States
(12) Patent Application Publication
(10) Pub. No.: US 2026/0337536 A1
(43) Pub. Date: Oct. 29, 2026

(54) Cultivating Aquatic Organisms Such as Algae, in Particular to Support the Conversion of CO2 into Biomass

(71) Applicant: A Healthier Earth Limited, London (GB)

(72) Inventors: Alastair Collier, London (GB); Martin Lynch, London (GB); Tom Kearney-Mitchell, London (GB)

(21) Appl. No.: 19/104,039

(22) PCT Filed: Aug. 16, 2023
(86) PCT No.: PCT/GB2023/052150

§ 371 Date: Feb. 14, 2025

(30) Foreign Application Priority Data
Aug. 17, 2022 (LU) LU102994

(51) Int. Cl. C12M 3/00; A01G 33/00; C12M 1/00; C12M 1/36

(52) U.S. Cl. CPC C12M 23/44; A01G 33/00; C12M 21/02; C12M 23/48; C12M 29/18; C12M 41/06; C12M 41/48

(57) ABSTRACT

There is described a modular biocultivation system (100) for cultivating photosynthetically active aquatic organisms, comprising a plurality of modules (100, 200, 300) for thin film cultivation, with each module comprising: a channel (102) for receiving an aqueous flow and organisms for thin film cultivation; and an artificial light source for illuminating organisms in the channel for photosynthetic activity; wherein the modules (100, 200, 300) are arranged above one another.

### FIELD OF THE INVENTION

The present invention relates to a modular biocultivation system for cultivating photosynthetically active aquatic organisms. The present invention also extends to modules for thin film cultivation of photosynthetically active aquatic organisms.

The present invention is particularly, but not exclusively, applicable to cultivating microalgae.

The present invention is particularly, but not exclusively, useable for supporting the conversion of CO₂ into biomass.

### BACKGROUND TO THE DISCLOSURE

Thin film biocultivation reactors generally involve illuminating a thin film of flowing liquid including a cultivated organism using sunlight to enable the organism to undergo a photochemical process. These reactors have many potential applications, including cultivation of algae which can allow the conversion of carbon dioxide (CO₂) to biomass, in turn providing a method of offsetting CO₂ emissions and reducing the amount of CO₂ in the atmosphere. Thus, these reactors can assist in combating climate change.

The present disclosure seeks to alleviate at least partially the problems of existing thin film reactors.

### SUMMARY OF THE INVENTION

Aspects and embodiments of the present invention are set out in the appended claims. These and other aspects and embodiments of the invention are also described herein.

According to an aspect of the disclosure, there is provided a modular biocultivation system for cultivating photosynthetically active aquatic organisms, comprising a plurality of modules for thin film cultivation, with each module comprising: a channel for receiving an aqueous flow and organisms for thin film cultivation; and a light source (preferably an artificial light source) for illuminating organisms in the channel for photosynthetic activity; wherein the modules are arranged above one another.

For ease of assembly each module may include interconnector features adapted for arranging each module relative to other modules to form an assembly of modules.

For robust and efficient construction the system may comprise a support frame adapted to receive the plurality of modules and/or the components of the modules.

For ease of access the support frame may be adapted to slidably receive the plurality of modules.

For stability the modular biocultivation system may further comprise means for mounting the support frame to a wall and/or ceiling and/or floor.

For modularity the system may comprise a plurality of stackable modules.

Each module preferably comprises a conduit arranged to provide a flow path from an end of the channel to a beginning of the channel for a flow circuit within the module. The conduit may be adapted for a fluid flow or a mass transport otherwise. The conduit may include a pump or a pumping means or a means of mass transport. The module may be adapted to enable a flow circuit within the module. The module may be adapted to enable a closed flow circuit or a flow circuit with any number of inlets and/or outlets. The flow circuit may include fluid flow and/or mass transport otherwise.

For efficient operation the modular biocultivation system may further comprise an adapted to mix a number of constituents to form an aqueous suspension of organisms for providing to one or more channels of the modules for cultivation.

For efficient operation the modular biocultivation system may further comprise a separator unit adapted to receive an aqueous flow from the modules, and to separate it into a suspension medium and organisms. The separator unit can serve to extract organisms from the aqueous flow. The separator unit may comprise a centrifuge, a filtration system and/or a sedimentation system. For efficient operation the modular biocultivation system may further comprise a purification unit for removing toxins and waste substances from the separated suspension medium from the separator unit, preferably wherein the purified suspension medium is provided to the modules or to a mixing unit for returning to the modules.

For enabling continuous operation a first subset of the plurality of modules may be adapted for providing first cultivation conditions (such as growth phase cultivation conditions), and a second subset of the plurality of modules may be adapted for providing different, second cultivation conditions (such as nutrient depletion phase cultivation conditions). The ratio of modules in the first and second subsets may be between 1:50 and 1:2, preferably 1:30 and 1:2, more preferably between 1:15 and 1:2. A portion of aqueous flow and organisms from the first subset of modules may be continuously provided to the second subset of modules.

For efficient environment control the modular biocultivation system may further comprise a sealed housing or envelope for the plurality of modules.

For efficient environment control the modular biocultivation system may further comprise a heating and/or cooling unit for heating and/or cooling the aqueous flow in the modules, and a controller for the heating/cooling unit. Temperature control is important for biological optimisation on one hand, and for CO₂ dissolution and storage capacity on the other hand.

For efficient conversion of carbon dioxide (CO₂) to biomass the organisms may be algae, preferably microalgae, further preferably saline microalgae and/or nannochloropsis.

For cultivation efficiency each module preferably further comprises means for causing the aqueous flow (and optionally organisms) to flow along the channel. The means for causing the aqueous flow to flow along the channel may include an inclination of the channel; a vibratory conveyor; a paddle; and/or a pump arranged to transfer the aqueous flow from an end of the channel to a beginning of the channel.

The artificial light source may include electrical illumination for illuminating the organisms in the channel. For high efficiency, cost-effectiveness and optimisation the electrical illumination may include light emitting diodes. Alternatively, or in addition to light emitting diodes, the electrical illumination may include: incandescent light sources, arc light sources, and/or gas-discharge light sources (e.g. plasma lamps).

For reduced power usage the artificial light source may include means for collecting sunlight and guiding it towards the organisms in the channel. The means for collecting and guiding sunlight may include light guides and/or mirrors.

For high cultivation efficiency the artificial light source for illuminating the organisms may be adapted to provide light at one or more discrete wavelengths.

For uniform illumination the artificial light source for illuminating the organisms may comprise a plurality of light outputs (e.g. a plurality of light emitting diodes). Preferably, the plurality of light outputs are arranged substantially parallel to the channel and/or distributed evenly relative to the channel and/or arranged equidistant to the channel.

The artificial light source for illuminating the organisms (in particular, the light outputs thereof) may be arranged above, below, and/or within the channel.

Each module may further comprise a means for cleaning the channel, preferably one or more of: a wiper adapted for wiping the channel bed, one or more nozzles for directing a fluid jet onto the channel bed, an ultrasonic cleaner unit, and/or an ultraviolet light source.

The system may comprise one or more reservoirs arranged to receive fluid (e.g. an aqueous flow) from the channel of one or more modules. Each module may include a reservoir, wherein the channel is adapted to direct the fluid/flow toward the reservoir.

The system may comprise means for injecting carbon dioxide to the aqueous flow in the modules. Preferably, the means for injecting carbon dioxide is adapted for dissolving carbon dioxide in the aqueous flow. Each module may comprise means for injecting carbon dioxide to the aqueous flow.

Each module may comprise a plurality of separate channel sections that are interconnected to form the channel. The channel sections may be arranged above one another. The module may further comprise means for redirecting the aqueous flow between channel sections.

The organisms may form a suspension and/or solution in the aqueous flow. Alternatively, the organisms may float on the aqueous flow.

According to another aspect of the disclosure, there is provided a module for thin film cultivation of photosynthetically active aquatic organisms, comprising: a channel for receiving an aqueous flow and organisms for thin film cultivation; and a light source (preferably an artificial light source) for illuminating organisms in the channel for photosynthetic activity; wherein the module is adapted for stacking on a further such module to provide a modular biocultivation system.

For ease of stacking the module may further comprise interconnector features adapted for arranging each module relative to other modules to form an assembly of modules.

For ease of stacking the module may include a load-bearing structure, preferably a frame, that is adapted for attachment, preferably reversible attachment, to the load-bearing structure of a further such module. The load-bearing structure may include legs adapted for engagement with legs of a further such module.

For efficient conversion of carbon dioxide (CO₂) to biomass the organisms may be algae, preferably microalgae, further preferably saline microalgae.

For effective cultivation the module may comprise means for causing the aqueous flow (and optionally organisms) to flow along the channel. The means for causing the aqueous flow to flow along the channel may include an inclination of the channel; a vibratory conveyor; a paddle; and/or a pump arranged to transfer the aqueous flow from an end of the channel to a beginning of the channel. The module preferably comprises a conduit arranged to provide a flow path from an end of the channel to a beginning of the channel for a flow circuit within the module. The conduit may be adapted for a fluid flow or a mass transport otherwise. The conduit may include a pump or a pumping means or a means of mass transport. The module may be adapted to enable a flow circuit within the module. The module may be adapted to enable a closed flow circuit or a flow circuit with any number of inlets and/or outlets. The flow circuit may include fluid flow and/or mass transport otherwise.

The artificial light source may include electrical illumination for illuminating the organisms in the channel. For high efficiency, cost-effectiveness and optimisation the electrical illumination may include light emitting diodes. Alternatively, or in addition to light emitting diodes, the electrical illumination may include: incandescent light sources, arc light sources, and/or gas-discharge light sources (e.g. plasma lamps).

For reduced power usage the artificial light source may further include means for collecting sunlight and guiding it towards the organisms in the channel. The means for collecting and guiding sunlight may include light guides and/or mirrors.

For high cultivation efficiency the artificial light source for illuminating the organisms may be adapted to provide light at one or more discrete wavelengths.

For uniform illumination the artificial light source for illuminating the organisms may comprise a plurality of light outputs (e.g. a plurality of light emitting diodes). Preferably, the plurality of light outputs are arranged substantially parallel to the channel and/or distributed evenly relative to the channel and/or arranged equidistant to the channel.

The artificial light source for illuminating the organisms (in particular, the light outputs thereof) may be arranged above, below, and/or within the channel.

The module may further comprise a means for cleaning the channel, preferably one or more of: a wiper adapted for wiping the channel bed, one or more nozzles for directing a fluid jet onto the channel bed, an ultrasonic cleaner unit, and/or an ultraviolet light source. The module may further comprise a reservoir, wherein the channel is adapted to direct the aqueous flow toward the reservoir.

The module may comprise a plurality of separate channel sections that are interconnected to form the channel. The channel sections may be arranged above one another. The module may further comprise means for redirecting the aqueous flow between channel sections.

The module may further comprise means for injecting carbon dioxide to the aqueous flow. Preferably, the means for injecting carbon dioxide is adapted for dissolving carbon dioxide in the aqueous flow.

The module may further comprise a pump adapted to provide the aqueous flow and organisms to the channel. For improved cultivation efficiency he means for injecting carbon dioxide may be arranged at, or adjacent, an inlet of the pump.

For efficient environment control the module may further comprise a heating and/or cooling unit for heating and/or cooling the aqueous flow in the channel, and a controller for the heating/cooling unit. Temperature control is important for biological optimisation on one hand, and for CO₂ dissolution and storage capacity on the other hand.

The module may be either adapted to provide first cultivation conditions for the organism, or adapted to provide different, second cultivation conditions for the organism; or adapted to provide either said first cultivation conditions or said second cultivation conditions. For a cultivation process with multiple cultivation phases, this can thus enable a first subset of modules to provide earlier phase conditions (e.g. growth phase cultivation conditions), and a second subset of modules to provide later phase conditions (e.g. nutrient depletion phase cultivation conditions), thus enabling continuous operation where different cultivation phases are desired.

According to another aspect of the disclosure, there is provided a module for thin film cultivation of photosynthetically active aquatic organisms, comprising: a plurality of channel sections for receiving an aqueous flow and organisms for thin film cultivation; and an artificial light source for illuminating organisms in the channel sections for photosynthetic activity; wherein the channel sections are arranged above one another.

The module may comprise means for redirecting the aqueous flow (and optionally organisms) between channel sections. The module may include features as aforementioned.

According to another aspect of the disclosure, there is provided a module or system for thin film cultivation of photosynthetically active aquatic organisms in a fluid, comprising: a sensor for sensing a concentration of organisms in the fluid; a light source (preferably an artificial light source) for illuminating organisms for photosynthetic activity; and a controller for controlling an illumination intensity of the light source in dependence on a concentration of organisms in the fluid.

The sensor may be an opacity or turbidity sensor, The sensor may be for sensing at a wavelength or in a wavelength range. The sensor may be for sensing reflectance, scattering (including forward scattering or backscattering) or transmissivity. The controller may be adapted to calibrate a sensor measurement in dependence on a prevailing illumination intensity of the artificial light source. The light source may be an electrical illumination for illuminating the organisms in the channel, or it may be a light guide or a mirror for providing sunlight to the organisms. The module or system may include features as aforementioned and/or be a module or system as aforementioned.

According to another aspect of the disclosure, there is provided a modular system for cultivating photosynthetically active aquatic organisms, comprising a plurality of modules for thin film cultivation as aforementioned.

The modules may be arranged above one another.

According to another aspect of the disclosure, there is provided a system for photosynthetic conversion of carbon dioxide into one or more biological products, the system comprising: a module for thin film cultivation of photosynthetically active aquatic organisms as aforementioned, or a modular biocultivation system as aforementioned; wherein one or more of the modules are adapted to receive carbon dioxide in the aqueous flow for photosynthetic conversion into one or more biological products.

The system may further comprise means for, at least in part, powering the artificial light source, preferably the means for powering comprising an electrical power source, more preferably a renewable electrical power source, yet more preferably powered by one or more of: solar, hydroelectric, wind, tidal, and/or biodiesel.

The artificial light source may be powered, at least in part, by burning of fuel (preferably a renewable fuel, more preferably biodiesel). The system may further comprise means for capturing, at least part of, exhaust carbon dioxide from said burning of fuel and for providing the captured carbon dioxide to the aqueous flow for photosynthetic conversion into one or more biological products.

The one or more biological products may include carbon and/or one or more biomolecules comprising carbon, preferably lipids.

According to another aspect of the disclosure, there is provided a modular photoreactor system for thin film photoreactions, comprising a plurality of modules for thin film photoreactions, with each module comprising: a channel for receiving a flow and reagents for thin film photoreaction; and an artificial light source for illuminating the reagents in the channel; wherein the modules are arranged above one another. The modular photoreactor system may include features of a modular biocultivation system as aforementioned.

According to another aspect of the disclosure, there is provided a module for thin film photoreactions, the module comprising: a channel for receiving a flow and reagents for thin film photoreaction; and an artificial light source for illuminating the reagents in the channel; wherein the module is adapted for stacking on a further such module to provide a modular photoreactor system. The module for thin film photoreactions may include features of a module for thin film cultivation of photosynthetically active aquatic organisms as aforementioned.

According to another aspect of the disclosure, there is provided a module for thin film photoreactions, the module comprising: a plurality of channel sections for receiving a flow and reagents for thin film photoreaction; and an artificial light source for illuminating the reagents in the channel; wherein the channel sections are arranged above one another. The module for thin film photoreactions may include features of a module for thin film cultivation of photosynthetically active aquatic organisms as aforementioned.

Any apparatus feature as described herein may also be provided as a method feature, and vice versa. As used herein, means plus function features may be expressed alternatively in terms of their corresponding structure.

Any feature in one aspect of the invention may be applied to other aspects of the invention, in any appropriate combination. In particular, method aspects may be applied to apparatus aspects, and vice versa. Furthermore, any, some and/or all features in one aspect can be applied to any, some and/or all features in any other aspect, in any appropriate combination. It should also be appreciated that particular combinations of the various features described and defined in any aspects of the invention can be implemented and/or supplied and/or used independently.

The invention extends to methods, system and apparatus substantially as herein described and/or as illustrated with reference to the accompanying figures.

As used herein, the term “cultivation” (and similar terms such as “biocultivation” or “cultivating”) preferably connotes growth process carried out by organisms, and may be a chemical process of particular interest carried out by organisms (such as photosynthesis) or a chemical process carried out by biochemically active substances (e.g. derived from organisms or synthetic).

As used herein, the term “artificial light source” preferably connotes a light source providing artificial illumination, preferably illumination that is not direct sunlight. Artificial illumination preferably includes electrical illumination that is powered by electricity (e.g. incandescent light sources, arc light sources, gas-discharge light sources including fluorescent lights, and LED light sources). Artificial illumination may include burning of a fuel, bioluminescence, chemiluminescence and other sources of light, whether or not electrically powered. Artificial illumination may also be provided in the form of light guides or mirrors suitably arranged to provide illumination from a remote light source, including sunlight.

As used herein, the term “thin film cultivation” preferably connotes cultivation of organisms in a thin film of fluid (preferably an aqueous flow). In some examples a suitable fluid film has a thickness of less than 20 mm or less than 10 mm, e.g. between 1 and 10 mm, or between 5 and 6 mm.

As used herein, the term “arranged above” preferably connotes an arrangement with at least part of a first module arranged vertically over a second module. Preferably at least part of a channel of a first module is arranged vertically over a channel of a second module.

It should be noted that the term “comprising” as used in this document means “consisting at least in part of”. So, when interpreting statements in this document that include the term “comprising”, features other than that or those prefaced by the term may also be present. Related terms such as “comprise” and “comprises” are to be interpreted in the same manner. As used herein, “(s)” following a noun means the plural and/or singular forms of the noun.

### BRIEF DESCRIPTION OF THE DRAWINGS

One or more aspects will now be described, by way of example only and with reference to the accompanying drawings having like-reference numerals, in which:

FIG. 1 is a schematic of a first module for thin film cultivation of photosynthetically active aquatic organisms;

FIG. 2 is a schematic of a second module for thin film cultivation of photosynthetically active aquatic organisms;

FIG. 3 is a schematic of a third module for thin film cultivation of photosynthetically active aquatic organisms;

FIG. 4 is a diagram of flows in a thin film cultivation module;

FIG. 5 is a schematic showing two modules of FIG. 3 connected in series;

FIG. 6 is a simplified schematic of a cultivation module in a modular biocultivation system;

FIG. 7 is a schematic of a modular biocultivation system with an array of cultivation modules;

FIG. 8 is a schematic showing a stacked array of cultivation modules;

FIG. 9 is a further schematic showing a stacked array of cultivation modules;

FIG. 10 is a schematic of the modular biocultivation system with further features;

FIG. 11 is a further schematic of the modular biocultivation system of FIG. 10;

FIG. 12 is a diagram of process flows in the modular biocultivation system of FIGS. 10 and 11; and

FIG. 13 is a schematic of a further modular biocultivation system with an array of cultivation modules.

### DETAILED DESCRIPTION OF PREFERRED EXAMPLES

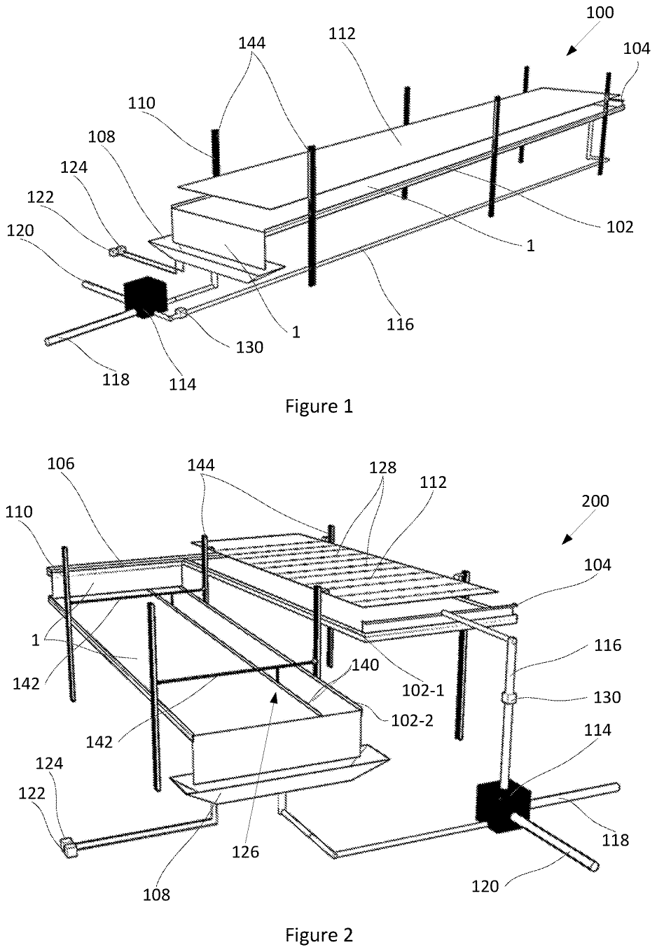

FIG. 1 shows a module 100 for thin film cultivation of photosynthetically active aquatic organisms (i.e. a first cultivation module 100). The module 100 includes a channel 102 for receiving an aqueous flow and organisms for thin film cultivation and an artificial light source in the form of electrical illumination 112 for illuminating organisms in the channel for photosynthetic activity. It will be appreciated that the artificial light source is primarily arranged to drive one or more photochemical reactions of the organisms (as opposed to e.g. heating the organisms).

Thin film cultivation is particularly well suited to cultivation of microalgae. Thin film cultivation is also well suited to cultivation of other organisms, such as duckweed, sargassum or cyanobacteria.

Thin film reactors are known and generally involve illuminating a thin film of flowing liquid using sunlight for efficient irradiation and enablement of photochemical processes, such as photocatalytic processing of waste water or, as here, photosynthesis in algae.

Microalgae such as nannochloropsis have been the subject of particular attention as they can be particularly effective at absorbing carbon dioxide (CO₂) and at producing biomass such as lipids and nutritional supplements that can be used further. Some nannochloropsis can accumulate high levels of polyunsaturated fatty acids. Conversion of CO₂ to biomass is of interest in the context of counteracting the release of CO₂ into the atmosphere from hydrocarbon fuels and other carbon-based materials. Photosynthesis can take advantage of sunlight as an abundant energy source for the fixing of CO₂. Schemes for cultivating algae for CO₂ fixing typically involve large open-air installations that can provide relatively large surface areas for exposure to sunlight. To provide optimal environmental conditions for cultivation not all geographical locations may be suitable. In particular large amounts of sunlight to illuminate the thin film and a steady water supply to replenish a suspension medium for the cultivated organisms can be beneficial. Otherwise convenient locations may not permit efficient cultivation.

It has been recognised that the use of sunlight for cultivation can afford some disadvantages, including the requirement for large areas for cultivation on a meaningful scale, challenges in open air systems where the environment can have a significant effect on cultivation, and geographical limitations if locations with both high sunlight availability and also availability of water are most suitable. In order to overcome these limitations a modular biocultivation system for cultivating photosynthetically active aquatic organisms has been developed. In the following first a single module is described in further detail, and then an assembly formed of a number of modules is described.

### Biocultivation Module

Thin film reactors are a bioreactor design for cultivating photosynthetically active aquatic organisms such as algae. These bioreactors typically use sloped channels to flow a thin layer of microalgal suspension (<10 mm thick) underneath a light source (typically natural sunlight). This exposure to light in a thin flowing film allows the algae to photosynthesise and reproduce at a faster rate than is typically experienced in horizontal ‘ponds’ or trays. The algae are suspended in a water based medium (fresh or salt water), the contents of which are controlled to promote specific mechanisms of algal cell behaviour, e.g. reproduction, or lipid synthesis. The thin layer of algal suspension allows improved light penetration and subsequent photosynthesis and therefore improved growth rates of algae cultivation.

These systems are recirculating, with the algal suspension flowing along the channel under the light source to a reservoir, where it is pumped back up to the top of the channel to flow underneath the light source again. Within the reservoir, the various parameters of the suspension can be recorded and the suspension can be topped up with various components as these are consumed by the growing algae or evaporate.

The principle is applicable to either batch or continuous flow. The algal suspension can be harvested, e.g. once it reaches a desired concentration. The system can be replenished with fresh suspension medium and seed algae. A system that would allow continuous operation rather than batch operation may be preferred.

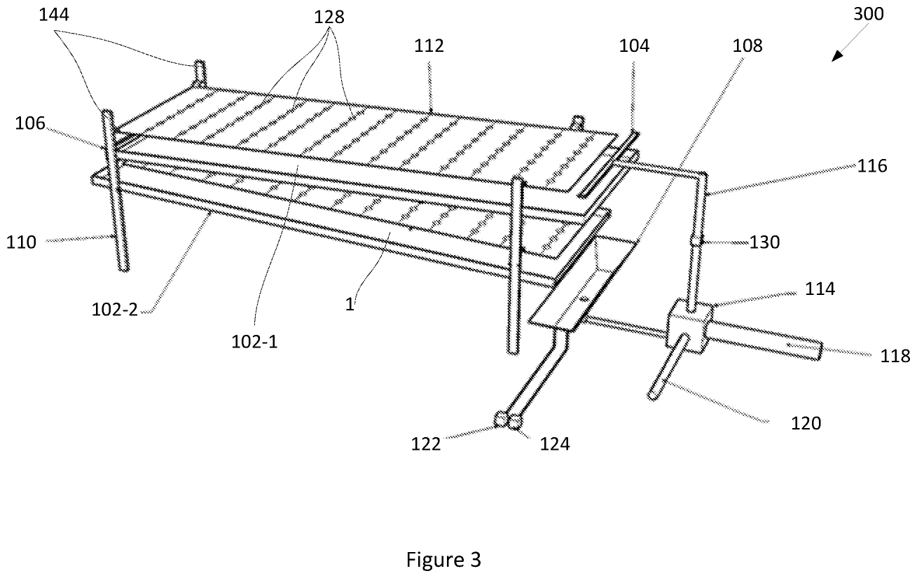

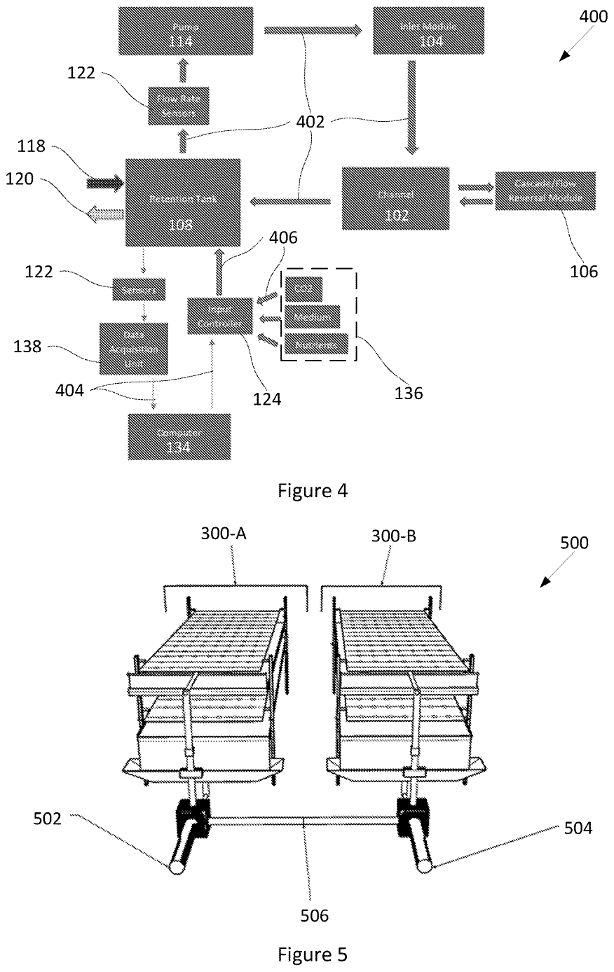

FIG. 1 shows a first thin film cultivation module 100. FIG. 2 shows a second thin film cultivation module 200. FIG. 3 shows a third thin film cultivation module 300. FIG. 4 shows a schematic of some of the material and energy flows 400 in modules 100, 200, 300. The same reference numerals are used in in each of FIGS. 1 to 4 to denote equivalent components.

FIG. 1 shows a thin film cultivation module 100 with a single channel section 102. FIG. 2 shows a thin film cultivation module 200 with channel sections 102-1, 102-2 arranged beside one another. FIG. 3 shows a thin film cultivation module 300 with channel sections 102-1, 102-2 arranged above one another. In FIG. 2 one of the channel sections 102-2 is shown without an electrical illumination panel 112 above the channel section 102-2; this is only so illustrated in order to show a cleaning unit 126.

The components of the thin film cultivation modules 100, 200 and 300 as shown in FIGS. 1, 2 and 3 respectively are now described. The components are generally selected to be corrosion resistant and cleanable. One or more of the components may be omitted in some variants. The components include:

Channels 102—channels 102 in which an aqueous flow and organisms (e.g. an algal suspension 1) flows along. In the illustrated example modules 100, 200, 300 the channel 102 is sloped, allowing gravity to create a flow of algal suspension 1. An exemplary channel inclination is 1.7%. In an alternative example, the module 100, 200, 300 includes other means for causing the algal suspension 1 to flow along the channel 102, such as a vibratory conveyor and/or one or more propellers. Using such other means for causing the algal suspension 1 to flow along the channel 102 (e.g. a vibratory conveyor) may allow the use of flat channels 102, thereby reducing the space occupied by (i.e. the height of) the module 100, 200, 300 and allowing more efficient stacking of multiple such modules 100, 200, 300 (as described in further detail below).

The channel 102 is where the algae is exposed to light using an electrical illumination panel 112. Channel 102 design is variable; examples include a simple plastic liner supported by a wire mesh or specially designed rigid piece channels 102 (metal or plastic). The channels 102 may include structures to influence the flow or the distribution of light in the algal suspension 1. The channel 102 is typically flat-bottomed so that a fluid film 1 of relatively uniform thickness is formed. The channel 102 may be selected wide enough for the intended flow to form a film of the intended thickness (e.g. 1-10 mm). The channel 102 is an open channel in the illustrated example such that the fluid film 1 is exposed to a gas environment in the region of the channel.

The channel 102 may for example be made of metal or plastic. Materials used are preferably capable of withstanding repeated temperature changes in the range of 5 °C 70 °C, in order to permit cleaning.

Flow-in unit 104—the flow-in unit 104 which distributes the algal suspension 1 onto the channels 102. The suspension 1 flows from the flow-in unit 104 onto the channels 102 via an overflow weir. The flow-in unit 104 slows the flow of the suspension 1 from a pump 114 and distributes it evenly over the entire channel 102 width. Baffles (not shown) may be used to slow and distribute the flow of suspension 1 within the flow-in unit 104. The flow-in unit 104 ensures a smooth flow at free fall from the flow-in unit 104 to the channel 102. The design of the overflow weir and beginning of the channel 102 are coupled to ensure this smooth flow.

Flow redirect unit 106—as shown in FIGS. 2 and 3, the flow redirect unit 106 provides means for redirecting the algal suspension 1 between channel sections 102-1, 102-2. The flow redirect unit 106 collects the algal suspension 1 from a first channel section 102-1 and redirects its flow onto a second channel section 102-2. The flow redirect unit 106 can redirect the algal suspension 1 to the following channel section 102-N horizontally and/or vertically (e.g. vertically for module 300, and horizontally and vertically for module 200) depending on the stacking design of channel sections 102-N. Cascades between channels and/or channel sections 102-N are generally designed to evenly spread fluid across the ‘output’ channel (e.g. channel section 102-2). It may be desirable to minimise sheer force on the algal suspension 1 at the cascade. Cascade design can be selected to promote or discourage aeration of the algal suspension 1. Channel outlets (e.g. between channel 102-2 and a reservoir 108, or between the flow redirect unit 106 and channel 102-2) can be designed with drip edges to smoothen the flow of algal suspension 1 and reduce foaming. Further, baffles can be included to optimise flow uniformity. It will be appreciated that the thin film cascade cultivation module 100 shown in FIG. 1 comprises a single channel 102 and so does not comprise a flow redirect unit 106.

Reservoir 108—the reservoir 108 collects the algal suspension 1 after it exits the channel 102. Within the reservoir 108, suspension 1 content can be measured and volumes taken. The reservoir 108 may be open (as shown in FIGS. 1 to 3); alternatively, it may include a closed portion forming a tank. A reservoir 108 may be provided for each module 100, 200, 300 as shown in FIGS. 1-3. Alternatively, multiple modules 100, 200, 300 may use the same reservoir (e.g. all modules 600 in system 700 shown in FIG. 7 may use one reservoir that collects the algal suspension 1 flow from all modules 600).

It will be appreciated that components 102, 104, 106, and 108 are arranged in a way so that there is no contact between them and the algal suspension 1 falls freely between them.

Structural frame 110—the load-bearing structure that holds the channels 102, electrical illumination panel 112 and other components in place. In the example modules 100, 200, 300 shown in FIGS. 1 to 3, the frame 110 comprises four legs 144. The legs 144 may, for example, be attached to the channels 102 and electrical illumination panels 112 via slots (not shown) in the legs 144 and corresponding protrusions from the channels 102 and electrical illumination panels 112. A wide variety of attachments can serve to attach the channels 102 and electrical illumination panels 112 to the legs 144.

Electrical illumination panel 112—the panel 112 comprises a plurality of light sources such as light emitting diodes (LEDs) 128 which provide light for algae photosynthesis. The LEDs 128 are evenly arranged across the panel 112 (e.g. in a series of rows as shown in FIGS. 2 and 3) to provide light evenly to the algal suspension 1 in the channels 102. In an example the maximum local illumination intensity at the algae is 1823 μmol m⁻² s⁻¹. The LEDs 128 may be configured to provide a light recipe for their output light, including spectral distribution and time variation which can be selected as appropriate. For some organisms uninterrupted illumination may be suitable, without a dark period simulating natural night conditions; this can increase cultivation efficiency (i.e. increase the amount of desired output per input resources, optionally over a given period of time).

In the example modules 100, 200, 300, the electrical illumination panel 112 is attached to the frame 110 and arranged above (and generally parallel to) the channel 102 such that the algal suspension 1 is evenly illuminated from above.

In an alternative example of module 300, electrical illumination (e.g. LEDs) for each channel 102 (e.g. channel 102-2) is attached to the bottom side of the channel 102 above (e.g. channel 102-1). To illuminate the ‘top’ channel 102 (e.g. channel 102-1), sunlight may be used and/or a separate electrical illumination panel 112 may be attached to the frame as shown in FIG. 3. Attaching electrical illumination to the bottom of the channels 102 can allow arranging the channels 102 closer to one another (as it removes the need for arranging a panel 112 between each channel 102), thereby reducing the height of the module 300 which can allow more efficient stacking of modules 300.

In a further alternative example, the electrical illumination panel 112 is arranged within the channel 102 (e.g. on or as part of the channel 102 bed) such that the algal suspension 1 is illuminated from below.

Pumps 114—pumps 114 are used to transport to the algal suspension 1 back up to the top of the cascade of channels 102. A variety of pumps may be used, such as magnetically coupled centrifuge pumps. Pumps can for example be selected to enable minimal sheer force and/or maximum pumping efficiency. An exemplary pump flow rate is 2.4 L/s.

Hoses 116—hoses 116 such as food safe PVC hoses which connect the reservoir 108, pump 114 and flow-in unit 104. The inner surface of the hoses 116 is preferably smooth so as to reduce build-up (e.g. of sediment and/or organic matter) in the hoses 116.

Flow rate sensor 130—used to measure the flow rate of the algal suspension between the pump 114 and flow-in unit 104.

Input Pipe 118—the pipe 118 through which the inputs to the channel 102 (e.g. inoculation medium and seed algae) are provided. Inputs may be provided at the channel 102 or elsewhere, e.g. at the reservoir 108. A number of different input pipes may be provided for different inputs, e.g. carbon dioxide, water, cultivation components.

CO₂ injection unit—a means (not shown) for injecting CO₂ into the algal suspension 1. Examples of a suitable injection unit include a perforated hose, bubble injection and/or pressure injection of CO₂. Dissolved CO₂ (CO₂(aq)) can be more easily absorbed by microalgae. Thus, injecting CO₂ into the aqueous suspension medium for dissolution of the CO₂ is preferred rather than merely providing the CO₂ to the cultivation environment otherwise. CO₂ is preferably injected in the form of a gas with a relatively high concentration of CO₂, for instance with a higher concentration of CO₂ than ambient air (ambient air typical CO₂ content: 0.04% by mole fraction or 420 parts per million). Examples of suitable gases with a relatively high concentration of CO₂ include exhaust gasses from combustion of carbon fuels. In some examples pure CO₂ or near-pure CO₂ may be injected. Alternatively the CO₂ is injected in the form of a gas with a similar or lower concentration of CO₂ than ambient air. The CO₂ may be injected in the form of ambient air. Advantageously, CO₂ injection is incorporated into the fall/suction position of reservoir 108 as it enters the hose 116 towards the pump 114 (i.e. as it enters a pumping mechanism); this can promote gaseous CO₂ to be entrained in the flow (dissolved form or as bubbles) rather than immediately escaping into the air, and thereby help ensure availability of the input CO₂ to organisms in the flow. CO₂ injection may be provided additionally or alternatively e.g. in the channel 102. A number of different CO₂ injection units may be provided for injection at different portions of the algal suspension 1.

Output Pipe 120—the pipe 120 through which the completed batch of algal suspension is output from the module 100, 200, 300.

Sensors 122—sensors 122 are used to measure various properties of the agal suspension 1 inside the reservoir 108. The sensors 122 may include one or more of the following: a volume flow rate sensor, a temperature sensor, a pH sensor, a volume sensor, and/or an algae concentration sensor (preferably able to act on a flowing suspension 1). It will be appreciated that a variety of specific sensors 122 for these purposes would be known to the person skilled in the art.

The sensors 122 may take readings for example every 10 seconds.

The module 100, 200, 300 preferably further comprises a data acquisition unit 138 (shown, e.g., in FIG. 4) which connects to all sensors and controllers and gathers data for passing to a computer 134.

Input controller 124—the controller 124 can allow suitably precise dosing of various elements of the algal suspension 1 inside the reservoir 108. The controller 124 may control the input to the algal suspension 1 of one or more of the following inputs 136:
CO₂—in an example, the input controller 124 comprises a mass flow rate controller adapted to control the output of a CO₂ injection unit;
Medium—for example, water—e.g. saline, fresh, as appropriate (e.g. for the cultivated organism and/or desired output products);
Nutrients—as appropriate; and/or
pH adjuster—as appropriate.

The controller may control further components in addition to inputs for ensuring the flow is suitable for the intended cultivation, for example to control a heater or cooler to maintain a desired temperature of the fluid. Temperature control of the fluid is important for biological optimisation on one hand, and for CO₂ dissolution and storage capacity on the other hand.

In an example the controller (or a separate lighting controller unit) is adapted to adjust the lighting in dependence on the algae concentration as sensed by suitable sensors. If the flow contains relatively highly concentrated algae then a more intense illumination may be provided than for a flow with a low concentration of algae. At lower algae concentration excessive illumination that might cause heating can be avoided. At higher algae concentration more intense illumination can enable all algae to receive sufficient illumination. For example an optical sensor can determine turbidity or opacity of the thin film as a measure of algae concentration (e.g. an infrared turbidity sensor), or reflectivity in a certain wavelength or range. A wide range of turbidity sensor arrangements may be adopted for algae concentration sensing (e.g. transmitted light sensing, nephelometric sensing including backscatter sensing, forward scatter sensing, and sensing at 90° to an incident light beam). Other sensors may be used to determine algae concentration, for instance impedance sensors, potentiometric sensors, or other optical or non-optical sensors. It will be readily apparent how a controller can control an electrical illumination to increase or decrease illumination strength based on algae concentration. Other light sources such as sunlight can also be concentrated to increase illumination or diverted elsewhere to reduce local illumination. As the different modules may contain algae populations at different stages of growth, this can permit a finite available light source such as sunlight to be used where it is most needed. In some examples the controller may be adapted to calibrate a sensor measurement for an illumination intensity. A momentary illumination intensity may affect an optical reading at a sensor, and a sensor measurement can be corrected or calibrated to prevent an undesired feedback loop in the control system.

The module 100, 200, 300 may further comprise a computing device/computer 134. The computing device 134 receives data from the data acquisition unit 138, and provides control inputs to the input controllers 124.

Cleaning unit 126—the (optional) cleaning unit 126 is used to clean the channels 102 between cultivation cycles which can allow reducing the time between cycles and increase overall production of the module 100, 200, 300. The cleaning unit 126 may for example be a mechanical or fluid-based system. An example cleaning unit 126 is shown in FIG. 2. The cleaning unit 126 comprises: a wiper 140 for wiping the channel 102 bed, and a support 142 attached to the structural frame 110 for supporting the wiper 140. The wiper 140 is movable along the support 142, thereby wiping the channel 102 bed. Optionally, the cleaning unit 126 comprises a cleaning substance dispenser, which may allow yet faster cleaning and further reduce ‘downtime’ of the module 200 between cultivation cycles.

In an alternative example, the cleaning unit 126 alternatively, or in addition, comprises one or more nozzles (e.g. high velocity/jet pressure nozzles) that release a fluid (e.g. water on its own or mixed with a cleaning substance) for cleaning the channel 102 bed.

In a further alternative example, the cleaning unit 126 furher comprises an ultrasonic cleaner unit that agitates a fluid (e.g. left-over suspension 1 or a specifically-provided fluid such as water) in the channels 102 to clean them.

It will be appreciated that the cleaning unit 126 may include both contact (such as the wiper 140 described above) and/or non-contact systems for cleaning the channels 102. For example, the cleaning unit 126 may include a non-contact system in the form of an ultraviolet (UV) light source for instance arranged above the channels 102 (e.g. in the electrical illumination panel 112) that acts as a UV steriliser and can be used for ‘bio-cleaning’ of the channels 102.

The third thin film cultivation module 300 design shown in FIG. 3, with channel sections 102-1, 102-2 arranged above one another, may be preferred over the first and second modules 100 and 200. Arranging the channel sections 102-1, 102-2 above one another can allow reducing the footprint occupied by the third module 300 as compared to a second module 200 of a similar size (i.e. of similar total channel 102 size), thereby allowing more efficient stacking (i.e. more modules per unit volume) of multiple such modules 300. The arrangement of the third module 300 can also allow reducing the length of the third module 300 as compared to a first module 100 of a similar size (i.e. of similar total channel 102 size).

Although shown in FIG. 3 as including two channel sections 102-1, 102-2, it will be appreciated that the third module 300 may likewise comprise three or more channel sections 102-N arranged above another. The number of channel sections 102-N may be selected depending on the desired overall channel 102 length. The length of the channel is suitably balanced against the requirement to replenish the flow, e.g. with CO2, which in the illustrated examples occurs at one position per cycle through the flow circuit. Other arrangements to replenish the flow are possible and the channel can be adapted accordingly.

The algal suspension 1 comprises a seed organism (e.g. algae) and a medium that supports the selected algae or other seed organism. In an example, the microalgal strain used for the seed culture is Nannochloropsis salina. In this example, the medium can include the following:
Inoculation medium (used at the start of cultivation);
   E.g. artificial sea water (ASW); it may be appropriate to omit CaCl₂ in the trace element solution;
Feed medium (used to replenish nutrients during an experiment); and
   E.g. ASW excluding NaCl MgSO₄ and CaCl₂

Salt water medium is preferred as it requires less freshwater, it has reduced contamination risk and it also contains more CO₂ in its aqueous phase due to the higher pH.

The flow of the algal suspension 1 in modules 100, 200, 300 takes the following route:
1. Starting at the pump 114, the algal suspension 1 is pumped, via the flow rate sensor 130, up to the flow-in unit 104, where it is evenly distributed onto the first channel section 102-1.
2. The suspension 1 then flows down the first channel section 102-1 to the flow redirect unit 106, where it is evenly redirected onto the next channel section 102-2. It will be appreciated that the number of channel sections 102-N and flow redirect units 106 may be modified, e.g. in dependence on the desired conditions within the algal suspension 1.
3. Once the suspension 1 flows down the last channel section 102-2, it falls into the reservoir 108.
4. Here the suspension 1 passes via the sensors 122 described above and enters the hosing 116.
5. Through the hosing 116, the algal suspension 1 reaches the pump 114 that returns it to the flow-in unit 104 and the process starts again.

An exemplary flow depth in the channel 102 is 5 mm or 6 mm. An exemplary flow velocity in the channel 102 is 0.4-0.5 m/s. A variety of suitable parameters for designing the channels 102 and associated components will be known to the skilled person for a particular organism.

Different growth behaviours or products can be produced from the seed organisms (e.g. algae) through exposing them to different environmental conditions. For example, depleting the medium of nitrogen encourages the production of lipids within the algae, an important feedstock for biofuel production. A plurality of the thin film cultivation modules 100, 200, 300 may be connected in series, to encourage the growth of algae (in a first one or more modules 100, 200, 300) and then the subsequent concentration of certain compounds within the biomass (in a second one or more modules 100, 200, 300).

FIG. 5 shows a cultivation module 500 comprising two of modules 300 connected in series. The cultivation module 500 comprises: module 300-A (e.g. configured for cultivation under growth phase conditions), module 300-B (e.g. configured for cultivation under nutrient depletion conditions), an input pipe 502 for providing seed algae and medium to module 300-A, a connector pipe 506 for transferring cultivated algal suspension 1 (e.g. following initial growth) from module 300-A to module 300-B for further cultivation (e.g. for lipid accumulation), and an output pipe 504 for providing an output suspension 1 that has been cultivated first in module 300-A and then in module 300-B. By providing different types of modules, each providing different cultivation conditions, the algae cultivation can be operated as a continuous process rather than by a batch process. A proportion of the flow is continuously diverted from module 300-A to module 300-B, and a proportion of the flow is continuously diverted from module 300-B for harvesting. In order to ensure the average residence time in the different modules is appropriately provided (e.g. 4 days in module 300-A and 15 days in module 300-B) the ratio of flows into module 300-A, between module 300-A and module 300-B, and out of module 300-B is selected appropriately and the ratio of number of module 300-A to number of module 300-B is selected appropriately.

FIG. 4 is a schematic process flow 400 diagram of some of the material and energy flows in modules 100, 200, 300 as described above. Arrows 402 in FIG. 4 indicate flow of the algal suspension 1 between the various components. The algal suspension 1 flows: from the reservoir 108 to the pump 114 (via flow rate sensors 122), then from the pump 114 to the flow-in unit 104, then from the flow-in unit 104 to the channel 102, then optionally to one or more further channel sections 102-N via one or more flow redirect units 106, finally returning back to the reservoir 108.

Arrows 404 in FIG. 4 indicate data transfer between the various components. The data captured by sensors 122 is transferred to the data acquisition unit 138 which aggregates the sensor data and transfers it to the computer 134. The computer 134 processed the sensor data and outputs control signals for the input controller 124.

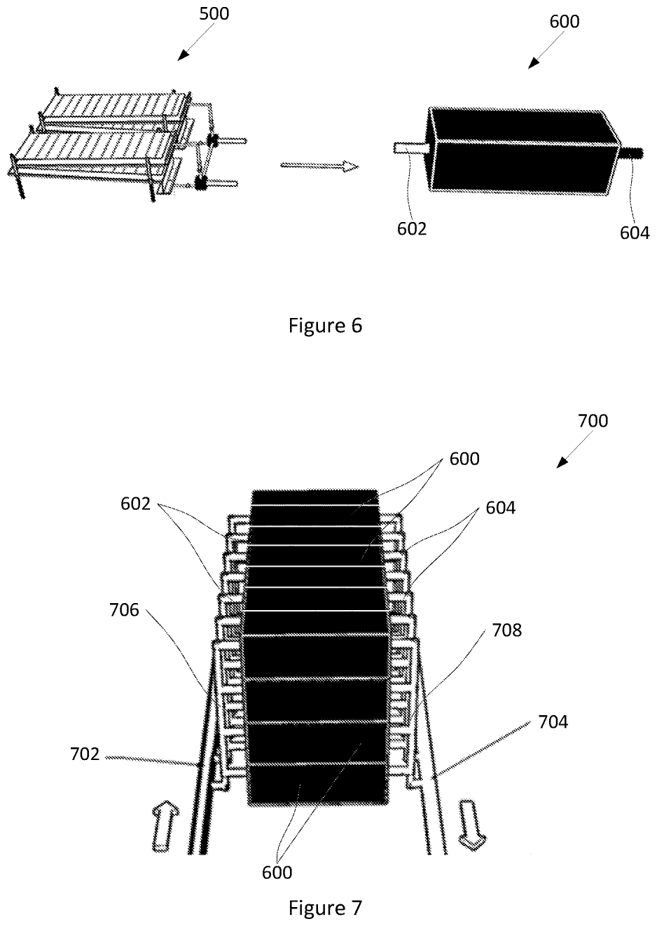

Arrows 406 in FIG. 4 indicate the flow of inputs 136 for the module 100, 200, 300. As shown in FIG. 4, example inputs 136 include: CO₂, medium, and/or nutrients. FIG. 6 shows a schematic of a cultivation module 600 in a modular biocultivation system. As shown in FIG. 6, the cultivation module 500 described with reference to FIG. 5 may be represented as a ‘black box’ cultivation module 600 comprising an input pipe 602, and output pipe 604. It will be appreciated that the ‘black box’ module 600 may represent any of the other above-described cultivation modules 100, 200, 300.

### Biocultivation System

FIGS. 7 to 12 show schematics of various modular biocultivation systems for cultivating photosynthetically active aquatic organisms. In a modular biocultivation system, a number of modules 600 as discussed above are arranged together. Individual modules 600 are arranged above one another.

Arranging modules 600 above one another might seem counterintuitive, as doing so obstructs illumination by sunlight for lower modules, and hence could be thought to prevent an important aspect of algae cultivation: to harness the energy from sunlight. The inventors have however recognised that, counterintuitive as it may seem at first glance, stacking can afford significant advantages and can still permit energy from sunlight to be harnessed at least indirectly.

In place of sunlight, an artificial light source is used in the modular biocultivation system. In particular, an artificial light source in the form of electrical illumination (e.g. using electrical illumination panels 112 in each module 600) may be used. Electricity is a well-developed energy carrier and ‘green’ electricity from renewable energy sources (including sunlight but also e.g. hydroelectric, wind, tidal, biodiesel, many of which are indirectly sunlight-powered) is becoming increasingly prevalent. By taking advantage of green electricity from renewable sources powering the electrical illumination need not counteract the overall aim of counteracting release of carbon dioxide CO2 into the atmosphere.

On the other hand, stacking can permit higher cultivation yield for a given footprint. A modular biocultivation system can be far more compact than a conventional system. Being compact can enable the installation to be housed in a building or envelope, which in turn can permit more effective environment control for the cultivation and reduced resource usage (e.g. by capturing and recycling evaporated water and released CO₂). Geographical location can become a less important factor to achieve high cultivation yield, facilitating the installation of a modular biocultivation system at a location that is convenient in terms of subsequent processing or raw material and energy availability, for example. Further, the electrical illumination can permit further independence of cultivation yield from environmental factors such as cloud cover and night/day cycle. In addition, the electrical illumination can allow providing light at one or more specific wavelengths, thereby promoting specific desired biocultivation processes whilst reducing heating of the algal suspension (as compared to sunlight providing light over a wide spectrum of wavelengths, many of which primarily provide heat energy but do not promote photosynthesis) thereby e.g. reducing evaporation of the medium and reducing resource usage (as medium can be topped up less frequently).

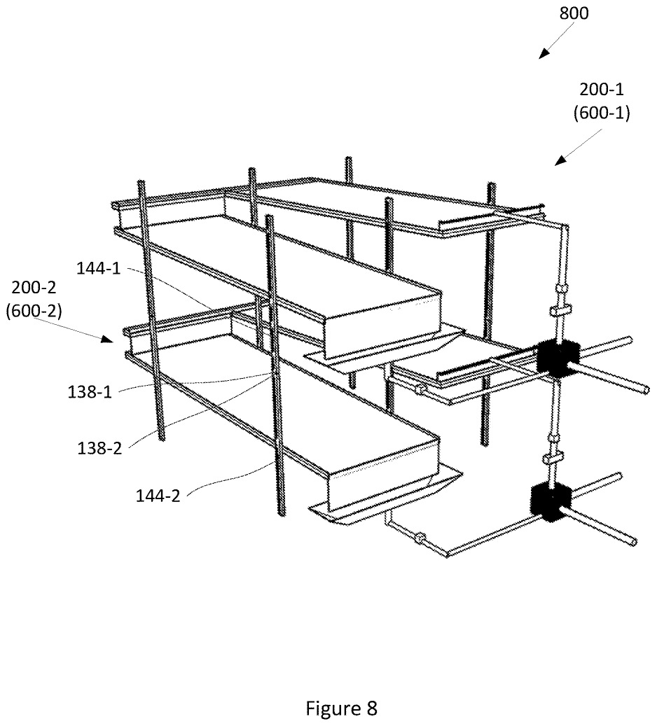

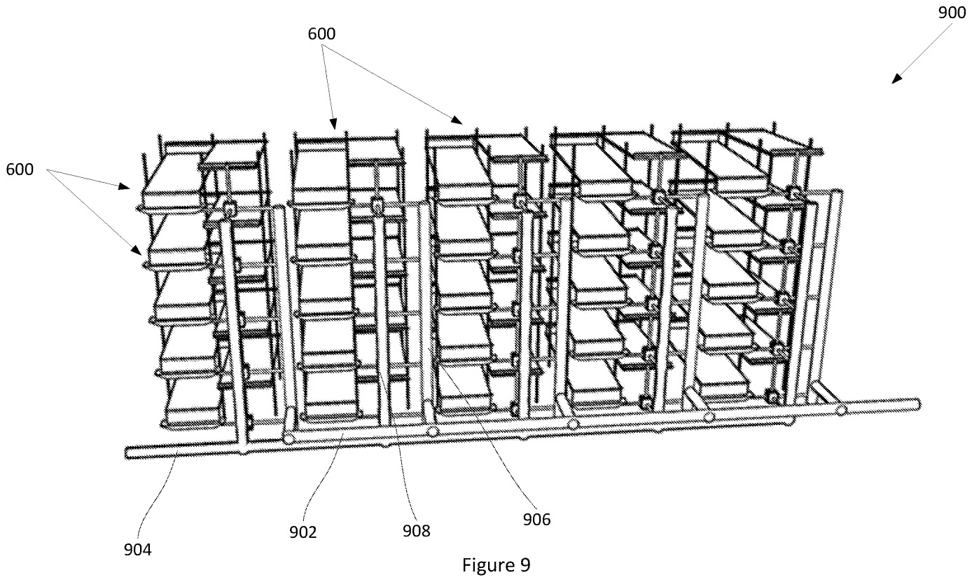

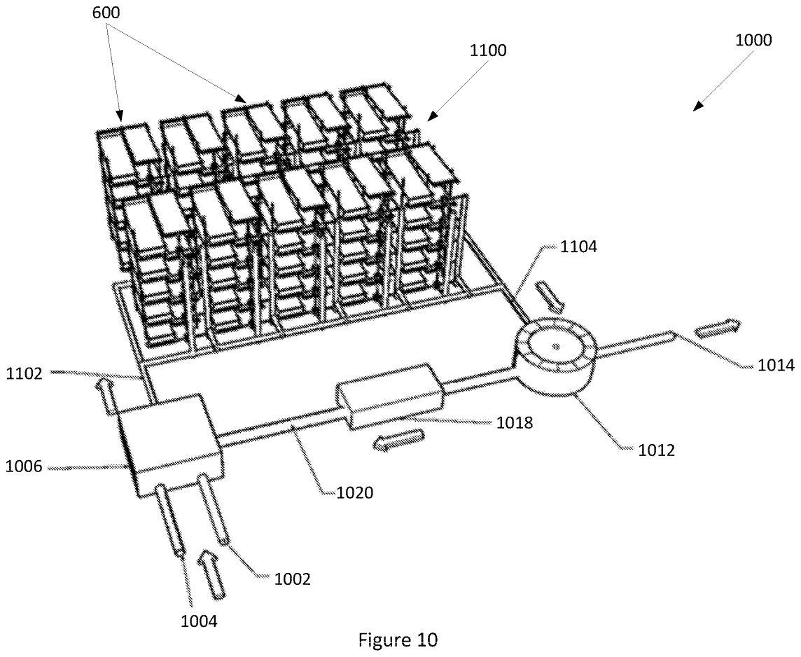

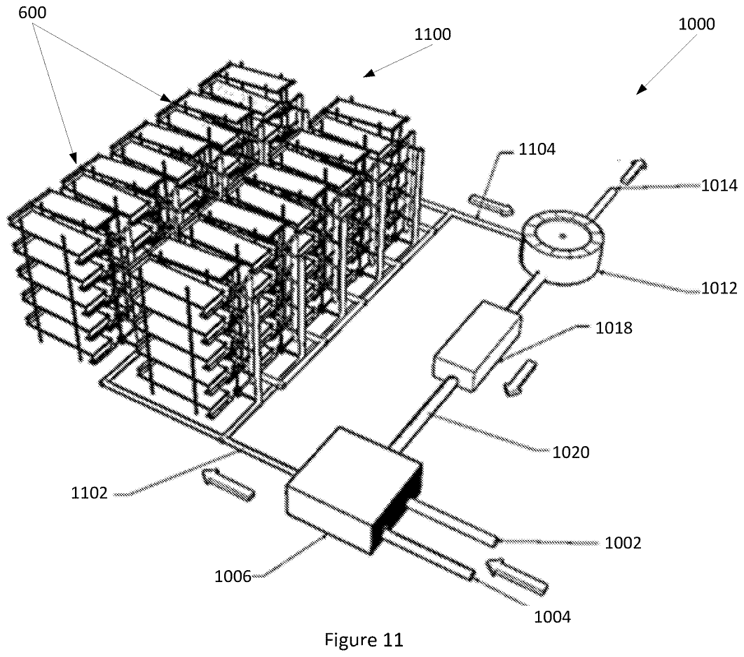

The modules 600 are stacked vertically, to maximise the cultivation area per square meter of floor space. In the example biocultivation system 700 illustrated in FIG. 7, four modules 600 are arranged above one another. In the example biocultivation system 800 illustrated in FIG. 8, two modules 200-1, 200-2 are arranged above one another. For ease of visualisation the electrical illumination is not shown in in FIG. 8. In the example biocultivation system 900 illustrated in FIGS. 9 to 11, five modules 200 are arranged above one another. In other examples, more or fewer modules 600 are arranged above one another; for example, between 2 and 20 modules or more may be stacked on one another. In the illustrated examples, the modules 600 are stacked in a grid with modules 600 squarely above one another; in other examples the modules 600 are laterally offset relative to upper or lower modules 600. The modules 600 in the biocultivation system may be arranged in: a single stack (see, e.g., biocultivation system 800 shown in FIG. 8), a grid—i.e. an array in 2 dimensions (see, e.g., biocultivation systems 700 and 900 shown in FIGS. 7 and 9 respectively), and/or a cube—i.e. an array in 3 dimensions (see, e.g., biocultivation system 1000 shown in FIGS. 10 and 11).

The arrangement of the modules 600 can be such that it optimises the use of space, materials and energy. An advantage of stacking the modules 600 is that it can allow maximising the biomass output per m³ for the biocultivation system. In turn, that can maximise the CO₂ absorption rate per m³.

In an example, the stacked modules 600 are contained in a large controlled environment (e.g. a warehouse). This can allow improved control of the environmental conditions (e.g. ambient temperature and humidity) and therefore of the cultivation processes in the modules 600.

FIG. 7 shows an example biocultivation system 700 comprising a 7×4×1 grid of modules 600 comprising seven stacks of four modules 600 arranged next to one another. FIG. 9 shows an example biocultivation system 900 comprising a 5×5×1 grid of modules 600 comprising five stacks of five modules 600 arranged next to one another. In the illustrated examples the biocultivation systems 700, 900 further comprise: a main input pipe 702/902 fluidly connected to the input pipes 602 of each module 600 via an input manifold 706/906, and a main output pipe 704/904 fluidly connected to the output pipes 604 of each module 600 via an output manifold 708/908. The main input pipe 702/902 provides input algal suspension 1 to the input pipes 602 of each module 600. In turn, the main output pipe 704/904 collects the output aqueous flow from the output pipes 604 of each module 600, e.g. for further processing. Suitable material and energy inputs, connections and outputs can be provided, and various adaptations can be made to the illustrated examples to provide the necessary materials and energy to each module.

In the example biocultivation systems 800, 900, 1000 shown in FIGS. 8 to 11, each module 600 is adapted for stacking on another module 600 to thereby form a stacked array of modules 600. As best seen in FIG. 8, the legs 144-1 of the frame 110 of each ‘top’ module 600-1 attach to the legs 144-2 of the frame 110 of the adjacent ‘bottom’ module 600-2. The legs 144-1, 144-2 each comprise corresponding interconnector features 138-1, 138-2 for engaging with one another. For example, one of the legs 144-1, 144-2 may comprise a protrusion (not shown), and the other of the legs 144-2, 144-1 may comprise a corresponding aperture (also not shown) that receives the protrusion thereby securing the legs 144-1, 144-2 (and modules 600-1 and 600-2) to one another. Corresponding interconnector features 138-1, 138-2 may be provided in all or only some of the legs 144 of each module 600. In another alternative example, the interconnector features of each module 600 comprise corresponding stacking cones for securing the modules 600 to one another.

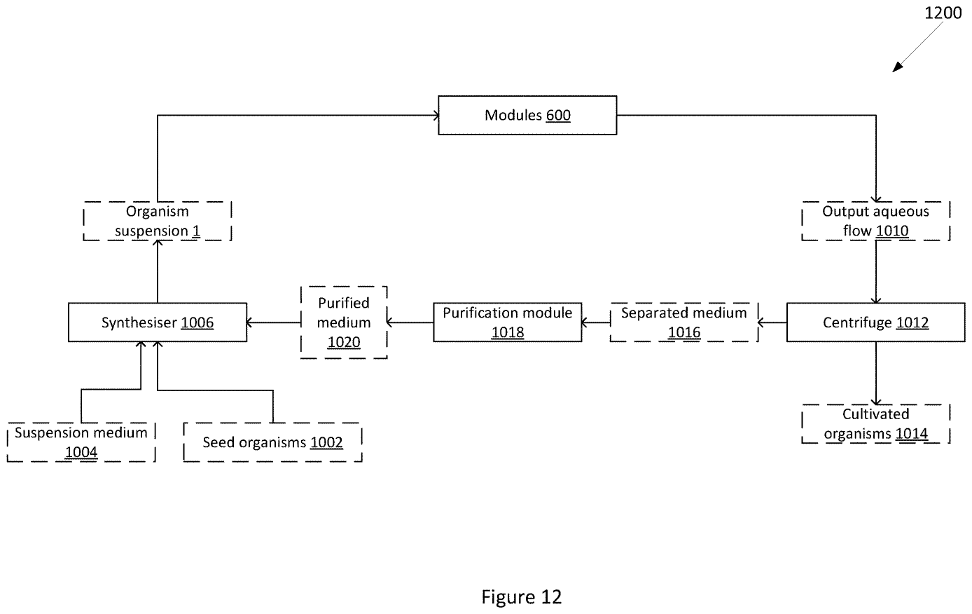

FIGS. 10 and 11 show schematics of a further example modular biocultivation system 1000 with further features as compared to the biocultivation system 900. FIG. 12 shows a diagram of process flows 1200 in the modular biocultivation system 1000 of FIGS. 10 and 11.

The biocultivation system 1000 comprises a 5×5×2 grid 1100 of modules 600 comprising five stacks of five modules 600 arranged in two rows next to one another. The biocultivation system 1000 further comprises: a mixing unit 1006, separator unit 1012, and a purification/recycling unit 1018. Further, similar to the biocultivation systems 700, 900, the biocultivation system 1000 also comprises a main input pipe 1102 for transferring the organism suspension 1 from the mixing unit 1006 to the modules 600, and a main output pipe 1104 for transferring the output aqueous flow 1010 from the modules 600 to the separator unit 1012.

In the example illustrated in FIGS. 10 and 11 a system mixing unit 1006 is included for producing a seed algal suspension 1 for feeding into the modules 600. The mixing unit 1006 receives various inputs—e.g. a suspension medium 1004 (e.g. fresh or salt water) and seed organisms 1002 (e.g. algae)—and mixes them into an aqueous flow/organism (e.g. algal) suspension 1 which is provided to the modules 600 for cultivation. While not specifically shown in FIGS. 10 and 11, further material (e.g. nutrients and CO₂) and energy (e.g. light and heat) are generally provided as the fluids travel through the modules for cultivation, as described above.

After the cultivation process in the modules 600 is completed, the separator unit 1012 receives an output cultivated aqueous flow 1010 from the modules 600 via the output pipe 1104, and separates the flow 1010 into cultivated organisms 1014 (e.g. biomass and other algae by-products) and separated suspension medium 1016 which can then be recycled to reduce resource usage of the system 1100. The cultivated organisms 1014 may be further transferred to a processing module (not shown) for further processing to obtain a desired output product. It will be appreciated that the separator unit 1012 may comprise multiple sub-units (e.g. multiple centrifuges) that perform the separation process in stages—e.g. each stage separating different parts of the cultivated organisms 1014 from the medium 1016.

In turn, the separated suspension medium 1016 is transferred from the separator unit 1012 to the purification unit 1018 which processes the medium 1016 such that the output ‘purified’ medium 1020 can be recycled back to the mixing unit 1006 (as shown in FIGS. 10 to 12), or directly to the modules 600 (e.g. to counteract evaporation of the medium 1004 from channels 102 of the modules 600). For example, the purification unit 1018 may process the medium 1016 to remove and/or disable toxins and waste produced during metabolism of the organism in the suspension 1.

A variety of suitable implementations of the mixing unit 1006, separator unit 1012, and purification unit 1018 will be known to the skilled person for a particular cultivated organism. For example, the separator unit 1012 may comprise one or more centrifuges, filtration systems, and/or sedimentation systems suitably arranged to separate the product (e.g. the cultivated organisms) 1014 from the suspension medium 1016.

In the example biocultivation systems 800, 900, 1000 shown in FIGS. 8 to 11, each module 600 comprises the thin film cultivation module 200 described above with reference to FIG. 2. It will be appreciated that each module 600 may instead comprise any of the other modules 100, 200, 500 described above.

### Alternative Examples and Embodiments

It will be understood that the invention has been described above purely by way of example, and modifications of detail can be made within the scope of the invention.

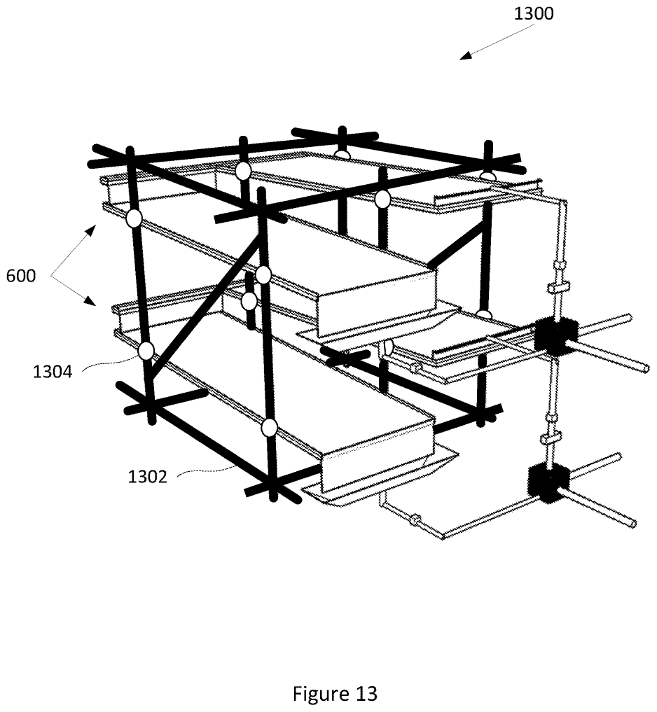

In an alternative example, the biocultivation system comprises a support frame adapted to receive the modules 600 and/or one or more of the above-described components of the modules (e.g. channels 102 and electrical illumination panels 112 individually). FIG. 13 shows an example of a biocultivation system 1300 where the support frame comprises a scaffold structure 1302 with a plurality of connectors 1304 for fixing the modules 600 and/or their components to the scaffold structure 1302. In another example the support frame comprises a warehouse shelving structure including a plurality of shelves that can receive the modules 600 and/or their components. The shelves may each comprise slots into which the modules (and/or their engagement features—e.g. protrusions on the sides of the modules 600) can slide in; this can simplify (re-)arrangement of the biocultivation system as well as other tasks such as maintenance, cleaning, or replacement of a module. To strengthen the support frame (or indeed the stacked modules 600 array as shown in FIGS. 7 to 12), the support frame and/or one or more of the modules 600 may be mounted (e.g. via angles) to a wall (and/or ceiling and/or floor) of the warehouse in which the system is located. The support frame may be mounted to the wall, suspended from the ceiling, free standing on the floor, or it may be attached to a building in any other way.

In another alternative example, analogous to the cultivation module 500 described above, the biocultivation system as a whole comprises a first subset of modules 600 configured to provide growth phase cultivation conditions, and a second subset of modules 600 configured to provide nutrient depletion phase cultivation conditions. The output cultivated organisms 1014 of the first subset of modules 600 are continuously provided as inputs to the second subset of modules 600 (directly or via mixing unit 1006). This can simplify the overall biocultivation process as the entire process (including the growth phase and nutrient depletion phase) can be operated continuously, without the need to modify conditions in each of the modules 600 to provide each phase. The first subset of modules 600 can continuously provide growth phase conditions, and the second subset of modules 600 can continuously provide nutrient depletion phase conditions. In some examples the growth phase takes less time, e.g. 1-10 days, than the nutrient depletion phase, e.g. 5-25 days. In this case the modules are selected such that the cultivation volume provided by the first subset is proportionally lower than the cultivation volume provided in the second subset to ensure that sufficient capacity is provided to the outputs of the first subset of modules 600 to allow the modules to operate at full capacity and enable continuous operation. For instance, the number of modules 600 in the first subset may be smaller than the number of modules 600 in the second subset, e.g. in a ratio between 1:50 and 1:2, e.g. between 1:10 and 1:30. For instance if a growth phase of 4 days is to be provided and a nutrient depletion phase of 20 days, then a ratio of 1:5 may be selected, with the modules linked in series. For instance if a growth phase volume output of 1 unit to a nutrient depletion phase volume output of 10 units is expected, then a ratio of 1:10 may be selected. Alternative or additionally, a module 600 in the first subset may each have a different capacity (e.g. channel width, flow path length, or other geometry) than a module 600 in the second subset; this can also enable organisms to remain for a first period under a first set of cultivation conditions and for a second period under a second set of cultivation conditions.

In another alternative example, one or more of the modules 600 in the biocultivation system comprises a housing that encloses the channels 102 and other module 600 components. This can allow yet more control of the environment in the channels 102 in the one or more module 600, which may be desirable for some cultivation processes. Each module may include its own envelope, as schematically suggested in FIGS. 6 and 7. All modules of a systems may be jointly housed in a common envelope. The modules may be provided in a house such as a building, with or without an envelope. A flexible film may provide a suitable envelope. The envelope or housing may be formed of rigid panels.

In another alternative example, the biocultivation system further comprises a turbine that collects energy from the flow of the algal suspension 1. For example, the system may comprise a turbine arranged in the output pipe 704/804/904/1104 to recover the potential gravitational energy from the algal suspension 1 (in particular, the algal suspension being output from modules 600 near the top of the stack).

While the biocultivation system described above has been discussed in the context of cultivation of organisms, it should be appreciated that other photoreactions such as waste water treatment may be implemented in a similar modular system. Adaptation of the modular biocultivation system for thin film photoreactions in general is straightforward. Suitable channels, electrical illumination, and ancillary features (inputs, outputs, upstream and downstream processing units) can be selected as appropriate for a specific photoreaction.

Where the means for causing the aqueous flow in the channel includes devices such as a pump arranged to transfer the aqueous flow from an end of the channel to a beginning of the channel, it should be appreciated that a wide variety of pumping means are suitable as a pump for transferring the aqueous flow from an end of the channel to a beginning of the channel. Pumping may be provided by pumping means including centrifugal pumps, positive displacement pumps, axial flow pumps, hydraulic ram pumps, ejector-type pumps, or other pumping means capable of transferring the aqueous flow (or a part of the aqueous flow, e.g. mainly the organisms) from an end of the channel to a beginning of the channel. As has been described above, the means for causing the aqueous flow in the channel may not include a pump; for example, this flow may be caused by using (e.g. vibrating) conveyors and/or propellors. Such means may be provided instead of, or in addition to, a pumping means.

While the above examples have shown the electrical illumination in the form of an electrically powered light source that is arranged above the channels, it should be appreciated that the electrical illumination may take other forms. For example, the electrical illumination may comprise light guides or mirrors suitably arranged to provide electrical illumination, with an electrical light source that is arranged elsewhere than illustrated in the figures. A wide variety of electrical light sources are known and available, including incandescent light sources, arc light sources, gas-discharge light sources including fluorescent lights, and LED light sources. Electrical light sources may be powered with electric energy derived from energy sources such as sunlight, hydroelectricity, wind energy, tidal energy, biodiesel, biochar or other chemical energy, or any other energy source.

Further, it should be appreciated that the electrical illumination 112 may be replaced and/or supplemented with other artificial light sources. For example, in addition to electrical illumination 112, the artificial light source may provide light obtained from burning of a fuel (e.g. from fuel combustion), bioluminescence, and/or chemiluminescence. In another example, the artificial light source includes light guides (e.g. optical fibers) or mirrors suitably arranged to collect sunlight and guide it towards the channels 102 to supplement electrical illumination 112 and reduce power usage. The artificial light source may also include a prism used to split the collected sunlight into its constituent wavelengths so that light at one or more specific wavelengths can be provided to the suspension 1.

While the above examples have shown the cultivated organisms as algae suspended in a medium and flowing along the channel 102 as a suspension 1, it should be appreciated that the organism may be arranged differently relative to the medium. For example, instead of forming a suspension, the organism (e.g. duckweed) may float on the surface of the medium and remain relatively stationary, with the medium flowing underneath it—e.g. such that the medium flow along the roots of the organism allows the organism to receive nutrients. For such stationary organisms, a scraping device may be used for removing/harvesting the organism from the medium.

Each feature disclosed in the description, and (where appropriate) the claims and drawings may be provided independently or in any appropriate combination.

Reference numerals appearing in the claims are by way of illustration only and shall have no limiting effect on the scope of the claims.

1. A modular biocultivation system for cultivating photosynthetically active aquatic organisms, comprising a plurality of modules for thin film cultivation, with each module comprising:
a channel for receiving an aqueous flow and organisms for thin film cultivation; and
an artificial light source for illuminating organisms in the channel for photosynthetic activity;
wherein the modules are arranged above one another.

2. A modular biocultivation system according to claim 1, wherein each module includes interconnector features adapted for arranging each module relative to other modules to form an assembly of modules.

3. A modular biocultivation system according to claim 1, wherein the system comprises a support frame adapted to receive the plurality of modules and/or the components of the modules.

4. (canceled)

5. A modular biocultivation system according to claim 3, further comprising means for mounting the support frame to a wall and/or ceiling and/or floor.

6. A modular biocultivation system according claim 1 wherein each module comprises a conduit arranged to provide a flow path from an end of the channel to a beginning of the channel for a flow circuit within the module.

7-9. (canceled)

10. A modular biocultivation system according to claim 1 wherein a first subset of the plurality of modules is adapted for providing first cultivation conditions, and a second subset of the plurality of modules is adapted for providing different, second cultivation conditions.

11. (canceled)

12. A modular biocultivation system according to claim 10, wherein a portion of aqueous flow and organisms from the first subset of modules is continuously provided to the second subset of modules.

13-16. (canceled)

17. A modular biocultivation system according to claim 1, wherein the organisms are one or more of: algae, microalgae, and saline microalgae.

18-25. (canceled)

26. A modular biocultivation system according to claim 1, wherein each module comprises a plurality of separate channel sections that are interconnected to form the channel.

27. A module for thin film cultivation of photosynthetically active aquatic organisms, comprising:
a channel for receiving an aqueous flow and organisms for thin film cultivation; and
an artificial light source for illuminating organisms in the channel for photosynthetic activity;
wherein the module is adapted for stacking on a further such module to provide a modular biocultivation system.

28. A module according to claim 27, further comprising interconnector features adapted for arranging each module relative to other modules to form an assembly of modules.

29. A module according to claim 27, wherein the module includes a load-bearing structure, that is adapted for reversible attachment to the load-bearing structure of a further such module.

30-44. (canceled)

45. A module according to claim 27, comprising a plurality of separate channel sections that are interconnected to form the channel.

46. A module according to claim 45, wherein the channel sections are arranged above one another.

47. (canceled)

48. A module according to claim 46, further comprising means for redirecting the aqueous flow between channel sections.

49-51. (canceled)

52. A modular biocultivation system according to claim 1,
wherein one or more of the modules are adapted to receive carbon dioxide in the aqueous flow for photosynthetic conversion into one or more biological products.

53. A modular biocultivation system according to claim 52, further comprising a renewable electrical power source for, at least in part, powering the artificial light source.

54. A modular biocultivation system according to claim 52, wherein the artificial light source is powered, at least in part, by burning of a renewable fuel; wherein the system further comprises means for capturing, at least part of, exhaust carbon dioxide from said burning of fuel and for providing the captured carbon dioxide to the aqueous flow for photosynthetic conversion into one or more biological products.

55. (canceled)

56. A module according to claim 27, further comprising a sensor for sensing a concentration of organisms in the fluid; and a controller for controlling an illumination intensity of the artificial light source in dependence on the concentration of organisms in the fluid.

57. A module or system for thin film cultivation of photosynthetically active aquatic organisms in a fluid, comprising: a sensor for sensing a concentration of organisms in the fluid; an artificial light source for illuminating organisms for photosynthetic activity; and a controller for controlling an illumination intensity of the light source in dependence on a concentration of organisms in the fluid.

58-59. (canceled)
